(19) United States
(12) Patent Application Publication
(10) Pub. No.: US 2010/0268154 A1
(43) Pub. Date: Oct. 21, 2010

(54) APPARATUS AND METHOD FOR AUTOMATING AN ENEMA WITH CONTROLLED DISTENSION

(75) Inventor: David J. Vining, Houston, TX (US)

Correspondence Address: DANN, DORFMAN, HERRELL & SKILLMAN, 1601 MARKET STREET, SUITE 2400, PHILADELPHIA, PA, 19103-2307, US

(73) Assignee: UNIVERSITY OF MARYLAND, BALTIMORE, Baltimore, MD (US)

(21) Appl. No.: 12/742,358

(22) PCT Filed: Nov. 11, 2008
(86) PCT No.: PCT/US2008/083115

§ 371 Date: May 11, 2010

Related U.S. Application Data
(60) Provisional application No. 60/987,665, filed on Nov. 13, 2007.

(51) Int. Cl. A61M 13/00

(52) U.S. Cl. 604/26

(57) ABSTRACT

An apparatus and method are provided for use in administering a rectal contrast enema to a patient to prepare the patient for a medical procedure. A rectal catheter may be provided to facilitate administration of the enema preparation to the patient. The rectal catheter may include a retention balloon. A delivery mechanism may be provided to urge the enema preparation into the patient. A vacuum may be provided to remove waste from the patient. A gas insufflator may be provided to insufflate the patient's colon for a medical procedure such as a scan, and more specifically, a virtual colonoscopy.

### PRIORITY CLAIM

This application claims priority to U.S. Provisional Patent Application No. 60/987,665, filed Nov. 13, 2007, the entire disclosure of which is hereby incorporated by reference.

### FIELD OF THE INVENTION

The present invention relates to an apparatus and method for administering an enema and distending a patient's colon in preparation for a medical procedure such as a scan.

### BACKGROUND OF THE INVENTION

Virtual colonoscopy (“VC”) is a technique used to screen for colon polyps, cancers, and other abnormalities. A patient's colon is prepared before conducting a VC scan to enable clear visualization of the colonic wall. Preparation generally involves cleansing the patient's bowel with a cathartic agent, distending the patient's colon, scanning the patient, and analyzing the acquired images.

Tagging of residual fluid and stool in the colon can be accomplished by the oral administration of a contrast agent such as low-concentration barium, gadolinium-based contrast agent, and/or water-soluble iodinated solution. Tagging makes it possible for a doctor to distinguish residual fluid and stool inside the colon lumen from colonic wall lesions. Oral administration of tagging agents has several drawbacks. For instance, oral agents must be administered 2 to 48 hours prior to a scan in order for the agent to transit from the patient's mouth to the patient's anus. Oral administration also exposes the stomach and small bowel to the tagging agent(s) which is not necessary for examination of the colon or particularly well-tolerated by the patient. Further, oral administration of contrast agents may not achieve uniform distribution throughout the patient's bowel, for instance, due to individual differences in bowel transit times among patients. Uneven distribution of contrast agents can inhibit the ability of the doctor or technician to distinguish residual liquid and stool from true colonic lesions. Additionally, even when computer-assisted detection/diagnosis (CAD) systems are employed to assist image analysis, residual untagged colonic liquid and stool can adversely affect the performance of the detection/diagnosis system.

The process of administering an enema has remained unchanged for over a century and often suffers from several drawbacks. For instance, rectal catheters, connecting tubing, and enema containers are often jury-rigged with multiple connections that are prone to separating and/or leaking, hence creating biohazard risks. An objective means of assessing the efficacy of administration of the enema to the patient is not available. Specifically, the doctor, nurse, and/or patient subjectively assesses the effectiveness of the delivery of the enema during the course of the procedure. Further, the doctor or nurse often experiences difficulty in controlling the concentration of pharmacologic agents added to an enema preparation when the enema preparation is prepared manually.

Accordingly, an apparatus for administering a rectal enema to a patient, including contrast agents and other pharmacologic agents, in conjunction with distention of the colon for preparation of a medical procedure such as a scan, wherein such procedure may be performed in an automated manner with elimination of biohazard risks, is desirable.

### SUMMARY OF THE INVENTION

In accordance with the present invention, an apparatus and method are provided for use in administering an enema to a patient, particularly for administering one or more agents such as gastrointestinal tract contrast agents and/or spasmolytic agents. Such agents may be used in conjunction with distention of the colon in preparation for a medical procedure such as a scan, particularly a virtual colonoscopy scan.

In accordance with the present invention, a rectal catheter may be provided. The rectal catheter may have one or more lumens. An inflatable retention balloon may be provided for forming a seal between the rectal catheter and the inner aspect of the patient's anus. The retention balloon may encircle a portion of the rectal catheter. Fluid such as gas and/or liquid may flow through a lumen located inside the catheter and into the retention balloon, thereby enabling inflation and deflation of the retention balloon. A retention balloon controller may be provided to controllably inflate and deflate the retention balloon, as well as to maintain a desired amount of inflation of the retention balloon throughout the medical procedure. Specifically, an inflator may be provided to inflate the retention balloon, and the inflator may be electronically and/or automatically controlled. A release valve may be provided to deflate the retention balloon, and the release valve may be electronically and/or automatically controlled.

An enema delivery mechanism may be provided for infusing an enema preparation into a patient, particularly via a rectal route of administration. Generally, the enema administered is a fluid, for instance, a liquid or a gas. The enema may be administered to the patient through one or more lumens of the rectal catheter. Optionally, an enema mixer may be provided to mix a pharmacologic agent, for example a contrast agent in a solid (e.g., powdered) or liquid form, with a carrier agent, for example sterile water, as a means to create a desired volume and concentration of enema to be delivered to the patient. Additional pharmacological agents may be administered to the patient in conjunction with the enema, either mixed with the enema, or delivered separately via a rectal catheter lumen shared with the enema delivery mechanism or via a separate rectal catheter lumen with its own delivery mechanism. The pharmacological agents may be administered either simultaneously during the enema administration or at a different time. Specifically, spasmolytic agents, bowel motility stimulating agents, antibiotics, probiotics, and/or replenishing electrolytes are examples of pharmacologic agents that may be administered. For instance, the enema mixer may combine one or more pharmacological agents, such as a contrast agent or a spasmolytic agent, with a carrier substance such as sterile water. Alternatively, a premixed enema preparation may be utilized. Optionally, the apparatus may also include a temperature control unit such as a heater to warm the enema preparation to a desired temperature to facilitate colonic relaxation. Alternatively, the temperature control unit may function as a cooling unit. All of these functions may be electronically or automatically controlled.

Optionally, a gas insufflator may be provided to administer gas to the patient's colon in order to distend it in preparation for a medical procedure such as a VC scan. Gas insufflation might occur before, during, or after administration of the enema. A gas insufflator may be provided to administer gas to the patient's colon following administration of the enema to distend the patient's colon in preparation for the medical procedure such as a VC scan. The gas insufflator may insufflate the patient's colon with a gaseous or aerosolized pharmacological agent to alter bowel physiology. For example, the pharmacological agent may promote colonic relaxation and distention. The gas and/or aerosolized agent supplied by the insufflator may flow through a lumen of the rectal catheter into the patient's colon. These processes may be electronically and/or automatically controlled.

A vacuum may be provided to suction waste, the enema liquid, and/or gas from the patient before, during, and/or after a medical procedure such as a VC scan. The vacuum may operate through a lumen of the catheter. Optionally, the vacuum may coordinate its operation with the enema delivery mechanism and/or the gas insufflator. For instance, the vacuum may intermittently suction gas from the patient's colon while the liquid enema is being administered. Alternatively, coordinated application of vacuum and gas insufflation may occur so that colonic pressure is increased in order to enhance removal of colonic waste. Coordinated application of vacuum and gas insufflation may also occur so that when elevated colonic pressure is encountered during gas insufflation of the colon, the gas insufflator will stop inflating and the vacuum will be activated to rapidly reduce colonic pressure to a desired level. These processes may also be electronically and/or automatically controlled.

The present invention may also include a processor in communication with one or any combination of the retention balloon controller, the enema delivery mechanism, the vacuum, and the gas insufflator to automatically control such operations, for example, under the control of a computer program. An input device may be provided to signal one or any combination of the retention balloon controller, the enema delivery mechanism, the vacuum, and/or the gas insufflator.

Optionally, the present invention may include an automated system and method of administering an enema in preparation for a medical procedure and for performing and controlling any of the above processes, functions or procedures.

### BRIEF DESCRIPTION OF THE DRAWINGS

The foregoing summary as well as the following description of the present invention will be better understood when read in conjunction with the figures in which:

FIG. 1 is a schematic diagram of one configuration of a rectal contrast enema and patient preparation system in accordance with the present invention.

FIG. 2 is a schematic diagram of one configuration of a rectal contrast enema and patient preparation system in accordance with the present invention.

FIG. 3 is a schematic diagram of one configuration of a rectal contrast enema and patient preparation system in accordance with the present invention.

### DETAILED DESCRIPTION OF THE INVENTION

Referring now to the Figures in general, wherein like reference numbers refer to the same components across the several views, there is shown an apparatus, generally designated 10, for use in a medical procedure such as a scan, or more specifically, a virtual colonoscopy (“VC”) scan. In general application, the apparatus 10 may be used to administer an enema or other agent in order to prepare a patient for a medical procedure. In specific application, the apparatus 10 may be used to administer a rectal contrast enema, to distend the patient's colon with gas, and/or to remove excess waste from the colon for a medical procedure such as a VC scan. The term “VC scan” as used herein includes imaging of a patient's abdomen and pelvis accomplished with computed tomography (CT), magnetic resonance imaging (MRI), positron emission tomography (PET) scans, as well as other types of imaging modalities. Generally, a VC examination involves several steps. First, the patient may ingest an oral cathartic agent to rid the colon of the majority of solid stool and to liquefy the fecal stream. Examples of oral cathartic agents that may be administered include but are not limited to polyethylene glycol and saline (e.g. sodium phosphate solutions). An enema 200 may be administered to further cleanse the patient's bowel. The second step of stool tagging may deliver one or more pharmacologic agents to the patient's colon to change the imaging characteristics of any residual liquid or stool not expelled during bowel cleansing. Stool tagging can be accomplished by an oral or rectal route of contrast agent administration. Following the enema 200, administered for further bowel cleansing and/or stool tagging, excess colonic residual tagged fluid may be removed from the patient to facilitate the third step of subsequent gas insufflation of the colon. Effluent may be removed by application of vacuum. The effluent may be collected in a closed containment system, and the container with its associated tubing and rectal catheter may be disposed of at the conclusion of the VC scan.

The patient's colon may be insufflated with gas to distend the colon to a specified degree to enable visualization of the colonic wall. Carbon dioxide is an example of one gas that may be used to distend the patient's colon, but other gases including room air, oxygen, xenon, krypton, and nitric oxide may be utilized. Other agents, including pharmacological agents for spasmolysis, may be administered to the patient at selected times during the enema and/or the insufflation steps to facilitate colonic relaxation and distention. The fourth step of a VC scan involves scanning the patient's abdomen and pelvis once the patient's colon has been distended to generate image data of the colon. In a fifth step of image analysis, a VC scan, two-dimensional (2D) and/or three-dimensional (3D) image data analysis is conducted to identify colon polyps, masses, or other abnormalities. The likelihood of obtaining reliable VC scan results increases when the patient's bowel is properly prepared, specifically, when the colon is cleansed, the residual fluid in the colon is tagged, and the colon is relaxed and optimally distended. The present invention provides an automated system and method for administering an enema, specifically a rectal contrast enema, to a patient in preparation for a medical procedure, such as a scan, particularly a VC scan.

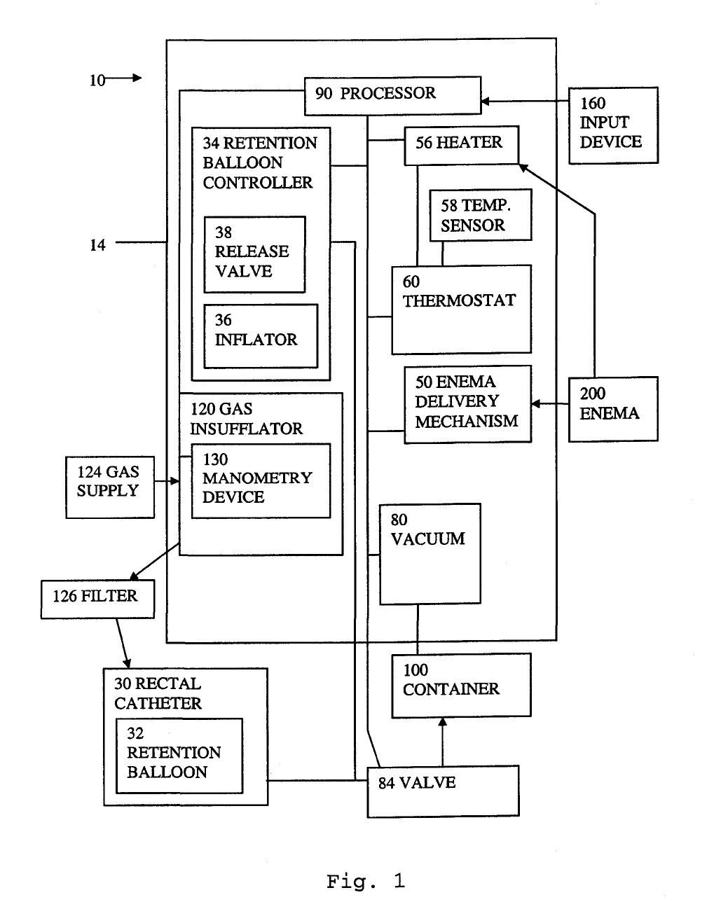

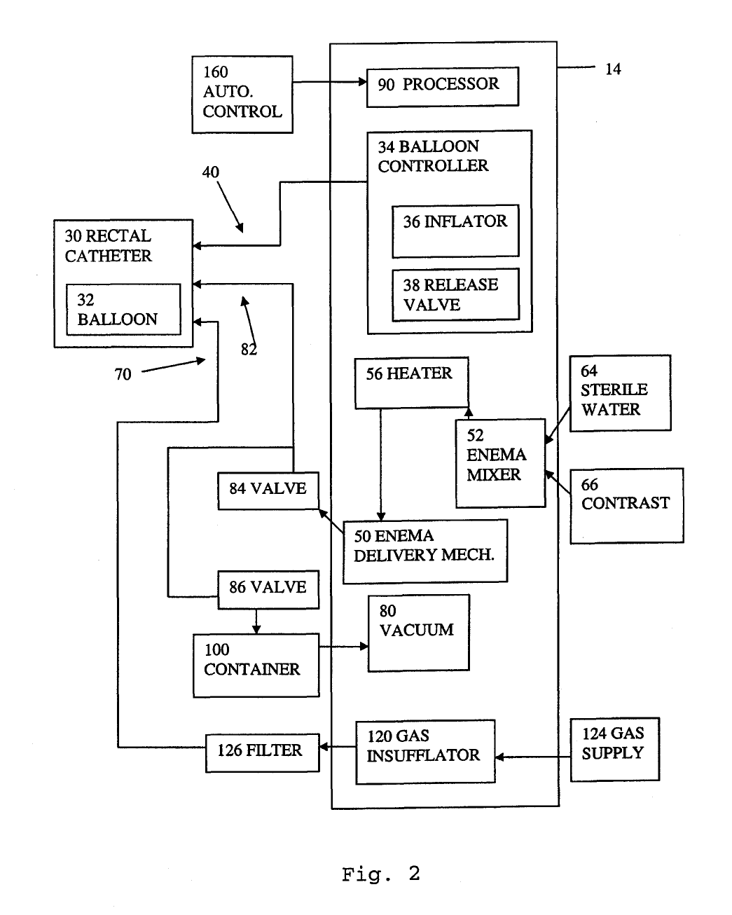

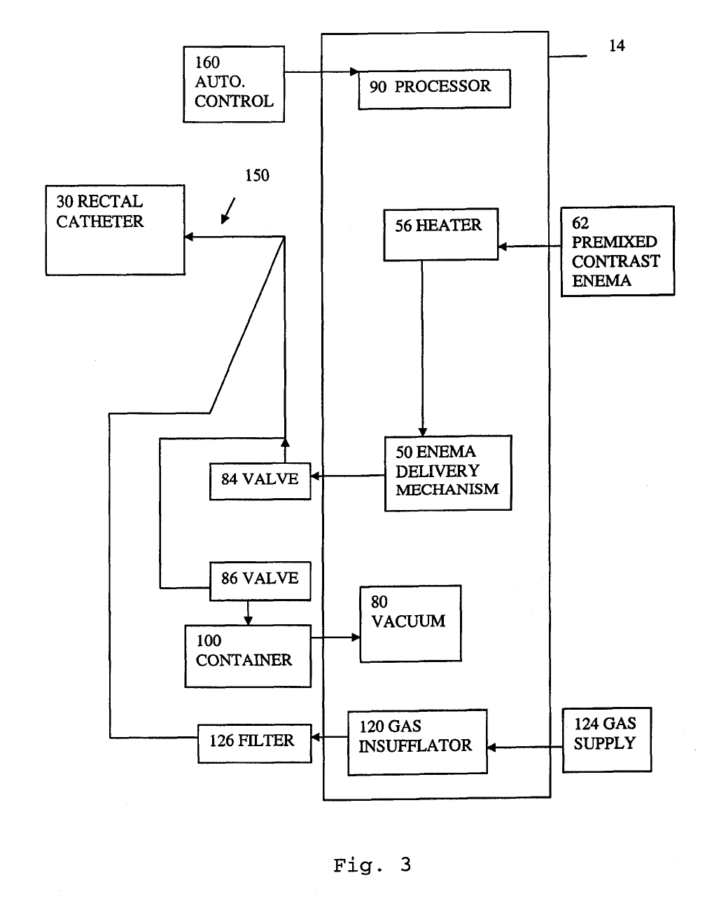

As shown in FIG. 1, the apparatus 10 includes a housing 14. A rectal catheter 30 is provided for communication with the patient. The rectal catheter 30 may be lubricated to ease the insertion of the rectal catheter through the patient's anus and into the rectum. One or more water-soluble lubricants such as K-Y® jelly or lidocaine jelly may be applied to the rectal catheter 30 to aid in its insertion into the patient. The catheter 30 may include one or more lumens in communication with the patient. For example, as shown in FIG. 2, three separate lumens 40, 70, 82, are provided. Alternatively, the rectal catheter 30 may have two lumens or simply one main lumen 150, as depicted in FIG. 3.

Optionally, the rectal catheter 30 may include a retention balloon 32 to assist in creating a seal between the catheter 30 and the patient's anus to prevent leakage of fluid and gas around the catheter to the outside environment. Optionally, the rectal catheter 30, retention balloon 32 and associated tubing may be constructed as a closed system (i.e., molded together) and made of disposable materials to provide for safe handling of potential biohazard waste. Once the rectal catheter 30 is inserted into the rectum of the patient, the retention balloon 32 is inflated to form a seal with the patient's anus to prevent fluid and/or gas from leaking out of the patient. The seal formed between the retention balloon 32 and the patient also hinders inadvertent expulsion of the rectal catheter 30 out of the patient. Gas or liquid may be used to inflate the retention balloon 32 in order to form the seal. For instance, gas or liquid may flow through a first lumen 40 of the rectal catheter, as shown in FIG. 2, and into the balloon.

Once the rectal catheter 30 is properly positioned inside the patient, the retention balloon 32 may be inflated or deflated to a selected degree. A retention balloon controller 34, such as a computer-controlled processor, effects inflation and/or deflation of the retention balloon 32 to a selected degree. Specifically, the retention balloon controller 34 communicates with an inflator 36 to inflate the retention balloon 32. If the retention balloon controller 34 receives input to deflate the inflation balloon 32, the retention balloon controller 34 communicates with a release valve 38 to deflate the retention balloon 32. For instance, deflation of the balloon 32 may vent gas directly to the atmosphere. Alternatively, deflation of the balloon 32 may vent gas or release liquid into a disposable container 100. The retention balloon controller 34 may monitor and maintain a selected level of inflation of the retention balloon 32 during the VC procedure and may function in response to inputs, such as manual, programmed or automated inputs, to inflate and deflate the balloon as desired. Inflation of the retention balloon 32 directed by the retention balloon controller 34 may increase the likelihood of establishing an effective seal between the retention balloon 32 and the patient throughout the medical procedure. Alternatively, external clamps may be provided to pinch the tubing closed that is connected to the retention balloon 32 closed after inflation so that the retention balloon 32 remains inflated to a selected degree throughout the procedure. Optionally, the rectal catheter 30 may have one or more sensors, such as pressure and volume sensors, to detect the degree of inflation of the retention balloon 32 and to signal the retention balloon controller 34 with data reflecting the degree of inflation of the retention balloon 32.

The retention balloon controller 34 may be controlled by a processor 90. For instance, an input device 160 may receive input data from a doctor or technologist through a manual input such as a keyboard or mouse. Alternatively, the input device 160 may also be voice activated, foot-switch activated, or any other similar activation means for hands-off control. Hand-off control is desirable so that the doctor does not need to change gloves after inserting the rectal catheter 30 or risk contamination of the apparatus 10 with dirty gloves. The input device 160 may also receive input data from hardwired circuitry, software, a data acquisition unit (DAQ), or other similar means. The input device 160 may transmit input data under the control of the processor 90. In general, the processor 90 may control the operation of one or any combination of the retention balloon controller 34, a gas insufflator 120, a vacuum 80, and an enema delivery mechanism 50. Alternatively, the input device 160 may transmit input data directly to the retention balloon controller 34, the gas insufflator 120, the vacuum 80, and the delivery mechanism 50. For instance, the input device 160 may signal the retention balloon controller 34, either directly or through the control of the processor 90, to control inflation or deflation of the retention balloon 32.

Once the rectal catheter 30 is inserted into the patient, an enema 200 may be administered. Prior to its administration, the enema 200 may need to be mixed and/or heated. One purpose of the enema 200 is to deliver a contrast agent throughout the colonic lumen to effect “stool tagging,” a means to change the imaging characteristics of the residual liquid and stool not eliminated during colonic cleansing, thereby permitting a doctor or technologist to distinguish residual liquid and stool in the images of the colon from true colonic lesions. Stool tagging may also improve the performance of computer assisted-diagnosis/detection (CAD) software programs that are used to interpret VC images. Accordingly, the enema 200 administered to the patient may include a contrast agent to achieve stool tagging. Unopacified residual fluid and stool may have similar x-ray attenuation (in the case of CT) or MRI intensity values (in the case of MRI) as that of polyps and solid colonic masses, and therefore it may be useful to alter the imaging properties of residual fluid and stool prior to conducting CT or MRI scanning to make residual fluid and stool distinct from true colonic lesions. A contrast agent may comprise solid (e.g., powdered), liquid, gaseous, or aerosolized form. A liquid form of contrast agent may include an iodinated-contrast solution such as iohexol. A gas form of contrast agent may include xenon or krypton gas. The contrast agent may be a positive contrast agent. A positive contrast agent will cause fluid and stool to appear brighter than soft tissue polyps and masses on a CT or MRI image. Examples of positive contrast agents when using CT as the imaging modality include barium, calcium, iodine and other similar substances. Alternatively, a negative contrast agent may be utilized. Negative contrast agents may cause fluid and stool to look darker on scan images. Negative contrast agents for CT scanning include fat-containing compounds. Further, an aerosolized contrast agent may be used. Aerosolized contrast agents may pass more readily than liquid agents throughout a patient's colon due to the tortuosity of some patients' colons. Contrast agents administered per rectum, whether in enema, gas or aerosolized form, may effectively and uniformly move throughout the colon in a matter of minutes.

The rectal contrast enema administered to the patient may include a premixed solution 62 having a carrier enema fluid, such as sterile water, mixed with a contrast agent, to achieve a specific concentration of contrast solution. For example, FIG. 3 shows a premixed contrast enema 62 being utilized. The premixed contrast enema 62 may contain specific amounts of the carrier enema fluid and the contrast agent to produce a specific concentration of the contrast solution. Alternatively, as shown in FIG. 2, the apparatus 10 may combine separate enema fluids and contrast agents in an enema mixer 52. For instance, sterile water 64, as the carrier fluid, and a contrast agent 66 may be combined and mixed. An exemplary contrast agent 66 includes a liquid contrast substance such as an iodinated agent (e.g., iohexol). The enema mixer 52 may mix together prepackaged volumes of sterile water with liquid contrast material. Other types of carrier liquids could also be used, such as normal saline, lactated Ringer's solution, or sodium phosphate solution. The contrast agent may be in a solid, liquid, gaseous, or aerosolized form. The input device 160 may signal the enema mixer 52 to start or stop mixing material. Alternatively, the processor 80 may function to control the enema mixer 52 to mix material.

A heater 56 may be provided to warm the enema to a selected temperature before administration to the patient. The input device 160 may signal the heater 56 and/or the thermostat 60 of a selected temperature for heating the enema 200. Alternatively, the processor 90 may communicate with the heater 56 and/or thermostat 60 to start or stop heating the enema preparation. The heater 56 may communicate with a temperature sensor 58 for detecting the temperature of the enema 200. If the temperature sensor 58 detects the temperature is not within a selected threshold, the thermostat 60 may signal the heater 56, which in turn may heat the enema 200. The heater 56 may heat the enema preparation to a temperature above that of normal body temperature (i.e., 37° C.) up to approximately 50° C., for example, to promote colonic relaxation. Once the sensor 58 detects the temperature of the enema 200 is within a selected threshold, the thermostat 60 may signal the heater 56 to shut off or to maintain such temperature range. As such, the thermostat 60 may be positioned separate from the processor 90. Alternatively, the thermostat 60 may be part of the processor 90. For instance, the thermostat may operate as a programmed routine of the processor or may be hardwired circuitry that compares a signal from the sensor with a selected programmed amount or with an input entered by a user. Once the enema preparation reaches a selected temperature, the enema 200 may be administered to the patient. In another application, the enema preparation may be heated before introduction into the apparatus 10.

A delivery mechanism 50 is provided to infuse the enema into the patient. The enema 200 may be infused into the patient through a lumen 82, 150 of the rectal catheter. If a multi-lumen catheter is utilized, the enema 200 may be administered through a lumen separate from that leading to the retention balloon 32, for example, through second lumen 82. The second lumen 82 may be larger than the first lumen 40 provided for the retention balloon 32 to facilitate flow-through. Alternatively, a single lumen catheter may be used and the enema may flow through the main lumen 150. The delivery mechanism 50 may comprise various mechanical devices to propel the enema preparation into the patient. In one embodiment, the delivery mechanism 50 may utilize a roller pump that squeezes flexible plastic tubing connected to the enema container in a repetitive and controlled manner to force the enema fluid in the tube to move forward in a direction towards the patient. In another embodiment, the delivery mechanism 50 may involve a compression device that exerts force against a container of enema preparation to propel the fluid from a container into the patient. For instance, a syringe injector may be utilized. In another embodiment, the delivery mechanism 50 may be configured to inject gas into a rigid container of enema preparation to force the enema into the patient. In yet another embodiment, positioning of an enema container at a height above that of the patient may be used to allow gravity drainage of the enema into the patient. The delivery mechanism 50 may include a means to control the flow of enema preparation into the patient. For example, a container of enema preparation may be positioned relative to the patient so that the enema preparation flows into the patient. The delivery mechanism 50 may control the rate of flow of enema preparation into the patient and/or the volume of enema preparation that is delivered to the patient. The delivery mechanism 50 may also control the pressure at which the enema is delivered to the patient. The delivery mechanism 50 may optionally include an aerosolizer to produce a spray-form of enema preparation or agent for administration into the patient. The aerosolizer may be under the control of the processor 90.

In a particular application, the delivery mechanism 50 infuses the enema through the rectal catheter 30 into the patient's colon at a selected pressure and/or flow rate. The input device 160 may signal the delivery mechanism 50 to administer the enema preparation to the patient or may signal the delivery mechanism 50 to cease enema administration. The input device 160 may signal the delivery mechanism 50 to reflect the rate at which the enema 200 may be administered to the patient. In one embodiment, the processor 90 may control operation of the delivery mechanism 50. A valve 84 may also be provided and set to an “open” position to direct flow of the enema from the delivery mechanism 50 into the catheter 30. The valve 84 may also be switched and oriented in a “closed” position to halt the administration of the enema 200 to the patient. In addition to mechanical valves, the valves may comprise a clamp or plunger that squeezes a section of tubing, thereby restricting flow of the enema.

Optionally, the delivery mechanism 50 may detect the amount of enema preparation and agent administered to the patient. Conversely, the vacuum 80 may detect the amount of fluid and gas collected from the patient. The processor 90 may also be used to controllably detect the amount of material administered to the patient and the amount of fluid and gas collected from the patient. For example, the amount of material administered to or collected from the patient may be detected by an automated switch, optical reading device, or determined by the doctor or technologist reading a visual scale on the enema container or a disposable waste container 100. Data reflecting the amount of material administered to and/or collected from the patient may reflect useful information to aid a doctor or technologist in patient care.

A vacuum 80 is provided to suction excess enema fluid and/or gas from the patient's colon. If the vacuum 80 is used in a configuration having a multi-lumen catheter, the vacuum 80 may utilize the second lumen 82 or even another lumen. If, however, a single-lumen catheter is provided, the vacuum 80 may operate through the main lumen 150. The input device 160 may signal the vacuum 80 instructions to activate or deactivate suction, or to change the rate or amount of negative pressure of suction. The processor 90 may control opening or closing the one or more valves 84 and 86. The processor 90 may also control the vacuum 80. The input device 160 may also signal the vacuum 80 to control the degree of negative pressure or suction to be applied to the patient.

Waste matter such as fluid and gas suctioned from the patient's colon may be collected in a disposable container 100. Desirably, the disposable container 100 should be sealed or easy to seal thereby providing safe biohazard handling and disposal. Optionally, a gas scavenger system may be provided in combination with the disposable container 100 to collect and process gaseous waste. The gas scavenger system may trap and dispose of hazardous gas, including gas inherent in the patient's colon before the start of the enema, thereby enabling a safer procedure. In one example, the rectal catheter, connecting tubing, and disposable container 100 may be constructed of disposable material as a continuous, sealed unit, thereby lessening the likelihood that the doctor or technologist would need to manually make one or more connections between each component of the system thus reducing or eliminating the risk of having the patient's body fluid contaminate the doctor, technologist, the apparatus 10, or the facility in which the examination is being conducted. Further, if the rectal catheter, tubing and container are a single unit, the doctor or technologist may not need to manually break one or more connections between the components to switch between enema delivery and gas insufflation. A single unit system thereby decreases the risk of biohazard contamination. In one example, a syringe injector or a roller pump may be used to administer an enema preparation into the closed unit system.

The apparatus 10 may provide for one or more valves 84, 86 to coordinate flow from the delivery mechanism 50 and to the vacuum 80. The valves 84, 86 may be electronically controlled. In one embodiment, an enema line and a vacuum line are connected by a Y-connector, and control is coordinated such that while the enema is infused into the patient the vacuum line is closed by a switchable valve. Further, the enema line may be closed by a switchable valve while the patient is suctioned. The input device 160 may signal valves 84, 86 to open or close. The valves 84, 86 may also be controlled by the processor 90. Optionally, the valves may be constructed of disposable material and designs. In one example, the valves may be positioned within the closed system but controlled externally relative to the closed system.

The apparatus 10 may provide for a gas insufflator 120 to distend the patient's colon with gas to a selected pressure for a medical procedure such as a scan. A gas supply 124 supplies gas to the gas insufflator 120. Gas flows from the gas insufflator 120 through the rectal catheter 30 into the patient. If a multi-lumen catheter is utilized, gas from the insufflator 120 may flow through a third lumen 70. If a single lumen catheter is utilized, gas from the insufflator 120 may flow through the main lumen 150. A hydrophobic filter 126 may be provided to prevent backflow of waste and bacteria into the insufflator 120. In addition, the valves 84, 86 for both the enema line and the vacuum line may be closed when the gas insufflator is activated to prevent gas from flowing into either of the enema or vacuum lines. The input device 160 may signal the gas insufflator 120 to administer gas to a patient at a selected rate. The processor 90 may also control the gas insufflator 120. Optionally, the gas insufflator 120 may include an aerosol delivery mechanism. For instance, the aerosol delivery system may be interfaced with the closed unit system described above. Alternatively, an aerosolized substance may be delivered to the patient independently from operation of the gas insufflator. Optionally, liquid is utilized to facilitate colonic distension. However, gas may be more desirable in selected applications since a gas-tissue interface provides for higher contrast and better visualization of the colonic wall than a fluid-tissue interface.

Optionally, a manometry device 130 is provided in communication with the gas insufflator 120. The manometry device 130 may function in concert with the enema delivery mechanism 50. The manometry device 130 may measure colonic pressure and gas/enema flow to the colon. The input device 160 may signal the manometry device 130 to deliver gas and/or enema within a selected threshold reflecting a selected colonic pressure and/or gas flow rate and/or enema flow rate. The processor 90 may also control the manometry device 130. The gas insufflator 120 may increase or decrease the amount of gas administered to the patient so that gas is administered to the patient within a selected threshold. The gas insufflator 120 may also increase or decrease the rate of gas flow into the patient so that gas is administered to the patient within a selected threshold. The gas insufflator may also be used to momentarily increase the gas flow/pressure that is administered, which may in effect create a boost of pressure. The gas insufflator 120 may communicate with the manometry device 130. If the volume of gas administered and/or the rate of gas flow and/or detected pressure is not within a selected threshold(s), the gas insufflator 120 then may adjust the amount of gas delivered and/or the rate of gas flow and/or gas pressure. The manometry device 130 may detect colonic pressure above a threshold to signal the need to start the vacuum 80 in order to rapidly vent the patient to relieve excessive pressure. The vacuum 80 may also be operated simultaneously or intermittently in coordination with the enema delivery mechanism 50 and/or the gas insufflator 120. For instance, the enema delivery mechanism 50 and/or gas insufflator 120 may be used to increase colonic pressure in conjunction with or immediately followed by suctioning generated by the vacuum 80 to facilitate fluid flow in the colon. In this instance, the orifice of the vacuum line at the end of the rectal catheter 30 may be positioned away from the opening of the enema line and/or the gas insufflator line to enable continuous flow (and to prevent the system from short-circuiting the fluid/gas flow). Optionally, the manometry device 130 may detect perceived patient pain as described in co-pending U.S. Patent Application Ser. No. 60/979,962, filed Oct. 15, 2007 entitled “Apparatus and Method for Use in Analyzing a Patient's Bowel,” incorporated herein by reference. Detection of perceived patient pain may be used to control various aspects of this apparatus.

The present invention also provides for a method to prepare a patient for a medical procedure such as a scan, particularly a VC examination. A bowel cleansing agent such as polyethylene glycol or saline cathartic (e.g. sodium phosphate) solution may be administered to the patient orally to cleanse the patient's colon of the majority of solid stool and to liquefy the fecal stream. A rectal catheter 30 may be lubricated and inserted into the patient's rectum. The rectal catheter may have a single lumen 150 as shown in FIGS. 1 and 3, or the catheter may have multiple lumens 40, 82, 70 as provided in FIG. 2. Further, the catheter may optionally include a retention balloon 32 as provided in FIG. 2. A doctor or technologist may lubricate the rectal catheter 30 for insertion into the patient by applying lubricant to the rectal catheter 30 tip. In one example, a water-soluble lubricant such as K-Y® jelly or lidocaine jelly may be applied to the rectal catheter 30. A seal formed between the rectal catheter 30 and the patient's anus may prevent leakage of fluid and gas from the patient.

In one configuration, the catheter 30 may include a retention balloon 32. The balloon 32 may be inflated to a selected amount to form a seal against the patient's anus. The retention balloon 32 may be inflated through a first lumen 40 in the rectal catheter 30 that is inserted into the patient's rectum. Once the rectal catheter 30 is inserted into the patient, a retention balloon controller 34 may control the inflation of the retention balloon 32 to a selected degree. The retention balloon 32 forms a seal against the patient's anus to prevent the catheter 30 from slipping out of the patient and to prevent leakage of fluid and gas. The input device 160 may signal the retention balloon controller 34 to inflate or deflate the retention balloon 32. The input device 160 may receive input data from hardwired circuitry, software, a DAQ, programmable circuitry, or other such means. Further, the input device 160 may receive input data from the doctor or technologist through a manual input such as a keyboard or mouse, or it may receive input from an automated switch, foot-pedal, or voice-activated microprocessor unit to enable hands-off control. The processor 90 may function to control inflation or deflation of the retention balloon. The retention balloon controller 34 may signal an inflator 36 to inflate the retention balloon 32. The controller 34 may also signal a release valve 38 to deflate the balloon 32 to a selected amount. Remote activation of the retention balloon controller 34 to inflate or deflate the retention balloon 32 decreases the likelihood the doctor or technologist would need to change gloves, receive assistance from a second person, or risk contamination if the doctor failed to change soiled gloves prior to retention balloon inflation.

Once the rectal catheter 30 and/or the retention balloon 32 have formed a seal with the patient's anus, an enema 200 may be administered. The apparatus may administer a contrast enema using a positive or negative contrast agent. In one example, the apparatus 10 may contain an enema mixer 52 as provided in FIG. 2 to combine two or more substances to form the enema 200 to be administered to the patient. The enema mixer 52 may utilize pre-packaged and/or pre-measured substances. Alternatively, the technologist may prepare the enema contrast solution by mixing powered or liquid iodinated contrast agent with approximately 1 to 2 liters water inside an enema bag. The processor 90 may record the amount of enema administered to the patient and the amount of waste (effluent) suctioned from the patient. Alternatively, the delivery mechanism 50 may record the amount of enema administered to and/or collected from the patient.

Another example, shown in FIG. 3, involves using a premixed enema 62. The premixed enema 62 may include substances having a specific desired concentration of contents. In one example, pharmacological agents, such as spasmolytic agents, may be administered to the patient as part of the enema 200 or via a separate lumen in the rectal catheter with an independent delivery mechanism, such as an aerosolized mechanism. In one example, pharmacological agents, such as spasmolytic agents may be administered to the patient as part of the enema 200. Optionally, other pharmacologic agents may be delivered to the patient's colon using this apparatus, including agents to treat conditions such as irritable bowel syndrome, bowel infections, bowel dysmotility, and bowel ischemia.

Optionally, the enema may be warmed by a heater 56 to promote colonic relaxation. The input device 160 may signal the heater 56 and/or thermostat 60 to warm the enema 200 to a selected temperature. Alternatively, the heater 56 and/or thermostat 60 may be controlled by the processor 90. In a particular application, the heater 56 communicates with a temperature sensor 58. The temperature sensor 58 senses the temperature of the enema 200. The thermostat 60 communicates with the sensor 58 and the heater 56. Specifically, the thermostat 60 may signal the heater 56 with information reflecting the temperature of the enema 200. If the temperature of the enema preparation is not within a selected threshold, the heater 56 may be activated to warm the enema preparation. If the temperature of the enema 200 is detected within a selected threshold, the heater 56 may shut off.

The enema is administered to the patient through the rectal catheter 30. If the catheter includes a single lumen, the enema 200 is infused into the patient through the main lumen 150 as shown in FIG. 3, and the vacuum and gas insufflator channels are closed. In one example, such as depicted in FIG. 2, the catheter may include multiple lumens in which case the enema may be administered to the patient through a second lumen 82. The second lumen may be larger than the first lumen 40. A delivery mechanism 50 propels the enema through the catheter 30 into the patient. The input device 160 may signal the delivery mechanism 50 to administer the enema preparation to the patient. The processor 90 may control the delivery mechanism 90. The delivery mechanism 50 may propel the enema 200 through the catheter 30 into the patient. Administration of the enema 200 to the patient may help distend the colon. The delivery mechanism 50 may also function to cease administration of the enema to the patient. During enema 200 administration, the patient may be positioned to promote fluid flow and even distribution of the enema throughout the patient's colon. For instance, the patient may be initially positioned in a left lateral decubitus position until the entire enema is delivered, and then turned to a right lateral decubitus position, a sitting up position, and/or a supine position to move the enema preparation throughout the patient's colon. Even distribution of the contrast agent increases the likelihood of obtaining reliable VC scan results. Further, uniform distribution of the contrast decreases the likelihood that unopacified water might obscure true colonic lesions.

Optionally, a manual balloon inflator may be used to deliver a momentary higher pressure to distend the patient's rectum if the patient experiences a collapsed rectum. For example, an optional manual balloon inflator may be used to distend a collapsed rectum detected by manometry during gas insufflation. Alternatively, an instruction could be sent by the controller to give a “boost” of gas insufflation by the gas insufflator to achieve the same goal.

The delivery mechanism 50 propels the enema 200 through one or more valves 84, 86 into the rectal catheter 30 and into the patient's colon. As the enema 200 is infused into the patient, the valve 84 may close the line leading to the vacuum 80. Once a selected amount of the enema has been delivered to the patient, the valve 84, or the second valve 86, is actuated by the processor 80 to shut the line to the delivery mechanism 50 and open the line to the vacuum 80. Optionally, the delivery mechanism 50 is deactivated.

Once the enema 200 has been delivered to the patient, excessive waste may be removed from the colon before a scan or other medical procedure is performed. For this purpose, the apparatus 10 may include a vacuum 80 to suction waste particulate matter, fluid, and gas from the patient. The vacuum 80 suctions through a lumen in the catheter 30 that communicates with the patient. If a single lumen catheter is utilized, the vacuum 80 may suction through the main lumen 150. If a multiple lumen catheter is used, the vacuum may suction through a second lumen 82. The vacuum 80 applies negative pressure suction to the patient's colonic lumen to remove excess enema effluent and other such residual matter, including solid, liquid, and/or gas contents. The vacuum 80 may be controlled by the processor 90 to activate or deactivate suction of the patient. The input device 160 may signal the vacuum 80 to activate or deactivate suction of waste from the patient. The processor 90 may also control the vacuum 80. Accordingly, the vacuum 80 may be activated after administration of the enema 200 to suction waste from the patient. The vacuum 80 may also be activated at the conclusion of a medical procedure to collect waste before removal of the rectal catheter 30 in order to empty the patient's rectum to prevent the patient from accidentally soiling the CT or MRI table.

One or more valves 84, 86 may be provided to coordinate infusion of the enema 200, gas insufflation, and suction of waste to and from the patient. For instance, a rectal catheter 30 having multiple lumens may be utilized so that the enema preparation may be administered to the patient at the same time that suction is applied. In a situation where the enema is administered while suction is applied simultaneously, the position of the orifice of the enema line is positioned at a distance from the orifice of the vacuum line to prevent a “short circuit” in fluid flow from occurring. In one embodiment, intermittent and/or alternating administration of the enema and application of vacuum may be applied to the patient. The input device 160 may signal the one or more valves 84, 86 to open or close. The one or more valves 84, 86 may also be controlled by the processor 90. The delivery mechanism 50 and vacuum 80 may share a lumen within the rectal catheter 30. As such, administration of the enema 200 should be coordinated with application of suction to the patient. In one example, two valves 84, 86 may be provided to coordinate actions of the delivery mechanism 50 and vacuum 80. A first valve 84 may regulate flow of the enema 200 from the delivery mechanism 50 into the catheter. A second valve 86 may be provided to regulate suction flow created by the vacuum 80 into the catheter 30. In one instance, “wall suction” available at most medical facilities, may be used to produce suction in connection with the vacuum 80. The valves 84, 86 may be activated such that while the first valve 84 is opened to permit flow of the enema 200 into the patient, the second valve 86 is closed to prevent flow into the disposable waste container 100. Alternatively, the second valve 86 may be activated to permit suction of the patient while the first valve 84 is closed. Such coordinated action of the valves 84, 86 may also be used to “vent” the patient during administration of the enema 200 (or during gas insufflation). Optionally, the enema may be administered to the patient by the delivery mechanism 50 at a higher pressure and/or volume than the force of suction applied by the vacuum 80 to the patient. If the patient's colonic pressure exceeds a selected threshold value, the first valve 84 may be closed, thereby stopping flow of the enema 200 from the delivery mechanism 50 to the catheter 30. The second valve 86 may then be activated to effect suctioning of the patient, thereby rapidly decreasing pressure within the patient's colon. Once the patient's colon has returned to a selected threshold range of pressure, the second valve 86 may be deactivated such that suctioning ceases. Optionally, the vacuum 80 may be deactivated. The first valve 84 may be activated so that administration of the enema 200 from the delivery mechanism 50 may resume. The amount of fluid and/or gas delivered to the patient, as well as the amount collected from the patient, may be measured and recorded.

Waste removed from the patient by the vacuum 80 may be collected in a disposable waste container 100. The disposable waste container 100 preferably is constructed as a closed system or has a closed seal to prevent any leakage of fluid or gas, thereby providing for a safe disposal means. Optionally, a gas scavenger system is provided for collection and disposal of gaseous waste. The scavenger system traps waste gas to prevent potentially hazardous gases from entering and contaminating the external environment. The rectal catheter 30, disposable waste container 100, and connecting tubing may be disposed together without the need to disconnect components that could potentially create a biohazard risk. Such items may be disposed of at the conclusion of the medical procedure without the need to disconnect components which could potentially increase biohazard risks.

If a detected colonic pressure is outside a selected threshold, the vacuum 80 may be activated to vent the patient into the closed containment system. If the vacuum 80 is utilized to vent the patient, the valve 84 may be activated to open the vacuum line and shut the enema delivery mechanism and/or gas insufflator lines. Optionally, the enema delivery mechanism 50 and/or gas insufflator 120 may be deactivated. The vacuum 80 may also be activated. Administration of the enema 200 is halted until the pressure has returned to a selected threshold. When the pressure returns to a selected threshold, the valve 84 may be actuated to open the line to the enema delivery mechanism 50 and to shut the line to the vacuum 80. Optionally, the vacuum 80 may be deactivated. The vacuum 80 may be activated at different times during the medical procedure. For example, the vacuum 80 may be activated at the conclusion of a scan to suction rectal fluid from the patient before the catheter 30 is removed to prevent the patient from soiling the CT or MRI table, or from soiling their own self. Suction may be coordinated with administration of the enema 200 to the patient. For instance, the vacuum 80 may be activated to remove waste from a patient and then deactivated so that the delivery mechanism 50 may resume administration of the enema 200 to the patient. As noted above, the vacuum 80 also assists in maintaining a selected colonic pressure. Activation of the vacuum 80 provides for rapid colonic decompression when the pressure within the colon exceeds a selected threshold. Accordingly, use of the vacuum 80 may be coordinated with deactivation of the enema delivery mechanism 50. Optionally, the enema delivery mechanism 50, vacuum 80 and the valves 84, 86 may be closed to prevent backflow of colonic gas and fluid from the patient's colon into the system, including when the system is idle.

Once the patient's colon has been cleansed of the majority of waste, a medical procedure such as a scan may be conducted. A gas insufflator 120 may be provided to distend the patient's colon with gas. A gas supply 124 provides gas to the insufflator 120, which in turn provides gas to the patient's colon. The insufflator provides gas to the patient's colon through a lumen in the catheter 30. If a single lumen catheter is used, the insufflator provides gas through the main lumen 150. If a multi-lumen catheter is used, the insufflator may provide gas through a third lumen 70 as shown in FIG. 2. The input device 160 may signal the gas insufflator 120 to administer gas to a patient at a selected rate, pressure, and/or volume. Alternatively, the processor 90 may control the gas insufflator 120. A hydrophobic filter 126 may be provided to prevent backflow of waste from the catheter to the insufflator 120. Optionally, the gas insufflator 120 may provide a manometry device 130 to measure the patient's colonic pressure and the rate as well as volume of gas flow into the colon. In one embodiment, the manometry device 130 and/or the processor 90 may include a pain sensor which detects pain perceived by the patient which may be used to activate venting of the patient using the vacuum 80, or other aspects of this device including the automated administration of pharmacologic agents, such as that used to achieve spasmolysis (i.e., colonic relaxation). The automated administration of pharmacologic agents could occur via an enema mechanism, aerosol mechanism, or gas insufflation mechanism. Optionally, a manual balloon insufflator device may be used to deliver a temporary higher pressure of gas to distend the patient's rectum if the patient experiences a collapsed rectum. Alternatively, the gas insufflator could be instructed to deliver this momentary boost of higher pressure gas.

The gas insufflator 120 may coordinate insufflation of the patient with the suctioning of waste from the patient by the vacuum 80. For instance, the patient may be insufflated with gas, such as carbon dioxide, from the gas insufflator 120 at the same time as waste is suctioned by the vacuum 80. This simultaneous action may counter the formation of a “vacuum lock,” whereby suctioning alone creates a negative pressure inside the colon equilibrating with the pressure produced by the vacuum to cease effective suctioning. The simultaneous action may also hinder the patient's rectum from collapsing. Alternatively, activation of the gas insufflator may be coordinated with deactivation of the vacuum 80. If the detected colonic pressure exceeds a selected threshold, the enema delivery mechanism 50 may cease administering enema to the patient. Optionally, the gas insufflator 120 may be deactivated to cease administration of gas to the patient. The patient may be vented, which may involve manual or automated means to reduce colonic pressure. For instance, if the colonic pressure rises during administration of the enema 200, a vacuum 80 may be utilized to apply negative pressure and suction to the patient's colon. Application of the suction may remove additional fluid and gas insufflated into the patient, thereby rapidly decompressing the patient's colon. One or more valves 84, 86 may be activated to facilitate suctioning of the patient's colon. Once the patient's colonic pressure returns to a selected range, the enema 200 may be delivered to the patient. Simultaneous activation of the vacuum 80 and gas insufflator 120 may avoid formation of a vacuum lock, wherein the efficiency of the vacuum 80 decreases.

Once a selected amount of gas is delivered to the patient, the patient may undergo a medical procedure such as a scan. Once the scan has been conducted, the doctor, technologist, an automated CAD program or other such device may analyze the scan results. Optionally, pharmacological agents may be administered to the patient by the enema delivery mechanism 50 or gas insufflator 120 at the conclusion of the procedure. The pharmacological agents may be administered to the patient using a separate delivery mechanism and line incorporated into the apparatus 10. The patient may be vented to release gas from the colon. The gas insufflator 120 is shut off and the first valve 84 is activated so an open line to the vacuum 80 is provided. The vacuum 80 may be activated to remove waste from the patient and collect the waste into a disposable container 100. Once the waste is removed, the retention balloon 32 is deflated. The rectal catheter 30 is removed from the patient. The disposable waste container 100 and connected tubing are discarded. Optionally, the rectal catheter 30 and associated tubing are also disposable and may be discarded along with the disposable waste container 100.

Optionally, gas may be administered to the patient before administration of the enema or delivery of a pharmacological agent or contrast agent to facilitate flow within a distended colonic lumen. For example, the patient's colon may be insufflated, and then an enema preparation may be administered to the patient's colon. The enema preparation may include pharmacological agents and/or contrast agents. Optionally, the patient's colon is distended before administration of an aerosolized contrast agent to enable distribution of the aerosol through the distended colon lumen.

Various steps of the above procedure may also be performed manually by the doctor and/or technologist.

It will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

1. An apparatus for preparing a patient's colon for a medical procedure comprising:
a. a multi-lumen rectal catheter for insertion into a patient, wherein a gas is administered to a patient's colon through a first lumen;
b. an inflatable retention balloon positioned along the rectal catheter for establishing a seal with the patient's colon, wherein fluid is supplied to the inflatable retention balloon through a second lumen;
c. a retention balloon controller for regulating an amount of inflation or deflation of the retention balloon;
d. an enema delivery mechanism for administering an enema preparation to the patient, wherein the enema delivery mechanism administers the enema preparation through a third lumen of the rectal catheter;
e. a vacuum for removing waste from the patient, wherein the vacuum removes waste through the third lumen of the rectal catheter; and
f. a processor in communication with one or any combination of the retention balloon controller, the enema delivery mechanism, and the vacuum.

2. The apparatus of claim 1, including a gas insufflator for inflating the patient's colon with the gas.

3. The apparatus of claim 1, comprising:
a. a heater for warming the enema preparation to a selected temperature for administration to the patient;
b. a temperature sensor for detecting the temperature of the enema preparation; and
c. a thermostat in communication with the temperature sensor and the heater for regulating the temperature of the enema preparation.

4. The apparatus of claim 1, wherein the processor communicates with and controls administration of at least one or more of:
a. the retention balloon controller to inflate the retention balloon;
b. the enema delivery mechanism to administer the enema preparation to the patient; and
c. the vacuum to remove waste from the patient.

5. The apparatus of claim 1, comprising an input device for at least one of remote activation or remote deactivation of at least one or more of:
a. the retention balloon controller to inflate the retention balloon;
b. the enema delivery mechanism to administer the enema preparation to the patient; and
c. the vacuum to remove waste from the patient.

6. An apparatus for preparing a patient's colon for a medical procedure comprising:
a. an enema delivery mechanism communicating with a rectal catheter for administering an enema preparation to a patient;
b. a vacuum communicating with the rectal catheter for removing waste from the patient;
c. a gas insufflator for inflating a patient's colon; and
d. a processor in communication with one or both of the enema delivery mechanism and the vacuum.

7. The apparatus of claim 6, including a rectal catheter for sealing a patient's colon from an external environment.

8. The apparatus of claim 6, comprising a mixer for mixing materials to form the enema preparation for administration to the patient.

9. The apparatus of claim 6, comprising:
a. a heater for warming the enema preparation to a selected temperature for administration to the patient;
b. a temperature sensor for detecting the temperature of the enema preparation; and
c. a thermostat in communication with the temperature sensor and the heater for regulating the temperature of the enema preparation.

10. The apparatus of claim 6, including at least one valve for coordinating administration of the enema preparation to the patient and application of the vacuum to the patient.

11. The apparatus of claim 6, wherein the processor communicates with and controls administration the gas insufflator.

12. The apparatus of claim 6, comprising an input device for at least one of remote activation or remote deactivation of at least one or more of:
a. the enema delivery mechanism;
b. the vacuum; and
c. the gas insufflator.

13. The apparatus of claim 6, wherein application of vacuum to the patient occurs simultaneously with insufflation of the patient's colon with gas.

14. An apparatus for preparing a patient's colon for a medical procedure comprising:
a. an enema delivery mechanism for administering an enema preparation to a patient, wherein the enema preparation includes a contrast agent;
b. a vacuum for removing waste from the patient; and
c. a processor in communication with at least one of the enema delivery mechanism and the vacuum.

15. The apparatus of claim 14, comprising:
a. a rectal catheter for insertion into a patient, wherein a gas is administered to a patient's colon; and
b. an automated controller for communicating input data to the processor.

16. The apparatus of claim 14, comprising a sealing means for sealing a patient's colon from an external environment.

17. An apparatus for preparing a patient's colon for a medical procedure comprising:
a. a rectal catheter for insertion into a patient;
b. an enema delivery mechanism for administering an enema preparation to the patient;
c. a vacuum for removing waste from the patient;
d. a gas insufflator for insufflating a patient's colon with a gas;
e. a manometry device for detecting selected parameters, wherein the selected parameters reflect an amount of insufflation of the patient's colon; and
f. a processor in communication with one or any combination of the enema delivery mechanism, the vacuum, and the gas insufflator.

18. The apparatus of claim 17, wherein the manometry device includes a pain transducer for detecting perceived patient pain.

19. The apparatus of claim 17, comprising an inflatable retention balloon positioned along the rectal catheter for establishing a seal with the patient's colon.

20. The apparatus of claim 18, including a retention balloon controller for regulating an amount of inflation or deflation of the retention balloon.
